(19) United States
(12) Patent Application Publication
(10) Pub. No.: US 2003/0040700 A1
(43) Pub. Date: Feb. 27, 2003

(54) Apparatuses and methods for providing IV infusion administration

(75) Inventors: Randall S. Hickle, Lubbock, TX (US); Samsun Lampotang, Gainesville, FL (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Scott Laboratories, Inc.

(21) Appl. No.: 10/208,184

(22) Filed: Jul. 31, 2002

Related U.S. Application Data
(60) Provisional application No. 60/378,046, filed on May 16, 2002.
(60) Provisional application No. 60/308,592, filed on Jul. 31, 2001.

(51) Int. Cl. A61M 31/00

(52) U.S. Cl. 604/67; 604/890.1

(57) ABSTRACT

An infusate cassette is described for use with an IV infusion system which controls the process of administering a drug to a patient. The cassette and other aspects of the infusion system may include disposable components, external redundant volume tracking, air removal and automated purge and prime capabilities, component removal lockout mechanisms, and/or redundant automated anti-free flow devices. An IV manifold comprising an imbedded high cracking pressure anti-free flow valve is also described for use with the infusion system. The cassette, IV manifold, and other aspects of the infusion system may be provided with quality assurance mechanisms for use with integrated IV infusion.

This application claims priority under 35 U.S.C. §119(e) to U.S. patent application Serial No. 60/308,592 filed Jul. 31, 2001 and to U.S. patent application Serial No. 60/378,046 filed May 16, 2002, both of which are incorporated herein in their entirety.

### FIELD OF THE INVENTION

The invention of this application relates generally to IV infusion of drugs to patients, and more particularly to aspects of an IV infusion system comprising an infusate cassette, an infusate container, and various quality assurance means.

### BACKGROUND OF THE INVENTION

Mechanically controlled infusion of a liquid drug from a reservoir directly to a patient is a useful process of administering a drug. An electro-mechanically controlled infusion process often provides a much steadier and more accurate administration of a drug or infusate than is possible from a human giving injections. By maintaining precise control of the flow rate of drug, an electro-mechanically controlled infusion device may ensure that the concentration of the drug in a patient's circulatory system remains steadily within the drug's therapeutic range.

Certain known medical devices for controlling the infusion of a liquid directly to a patient utilize pumping mechanisms to deliver liquid drugs from a reservoir such as a syringe, a collapsible bag, or a drug container to a patient supply tube. One example of such a device, shown in U.S. Pat. No. 6,186,977, includes a liquid drug supply in a collapsible bag and an infusion pump, which draws the drug directly from the supply and moves it along a flow passage to a patient supply tube.

Certain of these medical devices further utilize drug pump cassettes, which provide a rigid housing and pressure plate that interact with the pumping mechanisms of the devices. These cassettes serve as intermediary devices between drug containers and patient supply lines. A typical cassette includes a passage, which is acted upon by the pumping mechanism of an infusion device to move the drug along to the supply line.

One example of a cassette for use with a drug pumping system, shown in U.S. Pat. No. 6,165,154, has a fluid passage and a collapsible pressure conduction chamber for generating a pressure gradient to move drug along the passage. Another example of a cassette, shown in U.S. Pat. No. 6,202,708, provides a large chamber for mixing a powdered drug with a liquid solvent. This cassette also includes a pressure plate, which supports a fluid flow passage against which a peristaltic pump may act to move the liquid along to a patient delivery tube.

Certain liquid infusion devices which provide means for removing air that has entered their flow passages are also known. However, these devices often require an inefficient purging process which in turn requires human intervention and/or knowledge of the exact internal volume of all of the liquid passages in the system in order to flush air from the passages without losing excessive amounts of the drug.

There are also known drug infusion systems which are provided with computers or controllers that can track the volume of the liquid infusate remaining in a container by tracking internal encoder counts within the pumping mechanism. A problem with tracking volume based on internal effects, though, is that if there is an inconsistency with respect to a component within the infusion device, the calculated volume of drug infused may be incorrect and yet would nonetheless appear to be consistent with the operation of the device.

There are further drawbacks to the efficiency and safety of all of the aforementioned devices. One such drawback is that the known drug infusion devices may not allow for a cost effective means of disposing of those elements which come in direct contact with the drug. It may be beneficial from a quality control and patient safety standpoint to replace those parts of a liquid drug infusion device which directly contact the drug upon the completion of each infusion process. Disposal and replacement provide an efficient means of starting each infusion process with clean components that are free from residual infusate remaining from an earlier infusion or from vectors for cross-contamination from the previous patient. Some parts of the aforementioned devices, such as the drug pump cassettes, may be large and bulky and so might be expensive and clumsy to replace after single-patient use.

Another drawback of the above devices is that certain of their components, such as the drug containers, cannot be replaced during an infusion process, i.e., while the pumping mechanism is active, without introducing air into the system. Air may also be introduced into the systems if these components are accidentally removed from the device during an infusion process. Air bubbles that are entrained into the flow passages of a direct-to-patient infusion system can be dangerous if introduced into the patient's circulatory system.

Deaths have resulted from erroneous delivery of potent pain killers such as morphine by infusion pumps. Thus, a means of controlling the infusion rate of a drug based on a measurement or inference of an effect of the delivered drug on the patient may be beneficial. Such a means of control may be especially desirable during outpatient, ambulatory, gastrointestinal, cardiac catheterization, imaging and other procedures at remote and/or minimally staffed or equipped locations such as, among others, office-based surgery, imaging, or dermatology suites and far-forward military medical outposts where anesthesia and analgesia are provided with the concomitant risk of loss of consciousness and apnea.

### SUMMARY OF THE INVENTION

The present invention addresses the aforementioned drawbacks of existing drug infusion devices by providing an infusion system with an infusate pump cassette that may include disposable components, external redundant volume tracking, air removal and automated purge and prime capabilities, component lockout mechanisms, and/or redundant automated anti-free flow devices. The term “infusion system” as it is used herein may denote a stand-alone infusion pump that is not necessarily integrated with patient monitoring.

It is a further object of the present invention to provide a computer assisted IV infusion system with single-patient use disposable components to prevent potential cross-contamination and infusate carry-over from a previous infusion to a same or different patient. Components of this aspect of the invention that may be disposable may include, among other items, infusate containers, infusion tubing, pressure plates, infusion line connectors, cassettes, anti-reflux valves, high cracking pressure valves, IV manifolds and vascular access devices such as, among others, IV needles, cannulae and catheters.

The infusion system of the present invention may form part of a larger infusion system for computer assisted infusate administration that may include EKG pads or skin electrodes, and oxygen delivery, gas sampling and respiratory apparatuses, responsiveness query devices, and semi-automated modulation of infusion rate based on measured or inferred effects on the patient. The EKG pads or skin electrodes, oxygen delivery, gas sampling and respiratory apparatuses and responsiveness query devices may be disposable. The term “infusion system” as it is used herein may denote an infusion pump integrated into a larger system that manages the administration of infusate based on data from patient monitoring devices.

The integrated computer assisted infusate administration system is applicable for use in, among others, sedation and analgesia and deep sedation procedures. An example of such a system could be the sedation and analgesia delivery system described in U.S. patent application Ser. No. 09/324,759 filed Jun. 3, 1999, the entirety of which is herein incorporated by reference. The sedation and analgesia system of application Ser. No. 09/324,759 includes a patient health monitor device adapted so as to be coupled to a patient and generate a signal reflecting at least one physiological condition of the patient, a drug delivery controller supplying one or more drugs to the patient, a memory device storing a safety data set reflecting safe and undesirable parameters of at least one monitored patient physiological condition, and an electronic controller interconnected between the patient health monitor, the drug delivery controller, and the memory device storing the safety data set; wherein said electronic controller receives said signals and in response manages the application of the drugs in accord with the safety data set. The safety data set, as referred to by the electronic controller, may further include data regarding proper values for the identification and/or sources of drugs, supplies, components or attachments including the disposables listed above. Such identification may also be made by reading data from quality assurance modules accompanying the disposables.

It is a further object of the present invention that some of the disposable components are integrated into a single-use cassette for the transmission of infusate from containers to the patient. The cassette may be affixed to the infusion system with a single-motion snap-on action. The cassette is of a form so that its components align in the correct orientation with the permanent components of the system upon the single-motion snap on action. For example, a portion of the delivery conduit is positioned at the active portion of a pumping mechanism on the infusion system when the cassette is fitted into place.

The present invention allows for the infusate container to be removed and replaced during a given procedure without requiring the user to purge the infusion line of air. An infusate container lockout mechanism is provided to prevent removal of the container while the pump is running. To prevent free flow, various redundant infusion line lockouts automatically close off the infusate flow lumen when the cassette is not inserted into the infusion system. The lockouts are provided to guard against the pumping mechanism transporting air to the patient and against the free uncontrolled flow of infusate by gravity feed to the patient. To prevent air from reaching the patient if the lockout mechanisms fail, an air in line (AIL) detector may be used as a back-up safety device.

The infusion system provides an efficient means of controlling the flow of infusate from an infusate container such as, among others, a vial, syringe or collapsible bag to a manifold connector where the infusate may be combined with an IV solution and/or other fluids before administration to the patient. Computer control allows accurate flow rates and precise control of those flow rates for infusion and purging procedures as well as automated purging without the need for the user to intervene or remember to purge the line. Flow rate accuracy, combined with knowledge of the internal volume in the IV infusion set (acquired, for example, via a quality assurance module associated with the set), ensures the conservation of expensive infusate such as propofol, which may be wasted during manual control of the same procedures.

The present invention further provides a cassette with a sheathed infusate container spike made of injection molded plastic with an automated free flow prevention feature. The spike remains sheathed if the cassette is not fully engaged with a mating surface of devices such as, for example, a pumping unit or a sedation and analgesia delivery system. In general, the infusate container will be upside down but the invention also contemplates the possibility of having the infusate container upright. The cassette of the present invention may include molded snap retainers or clips integral to the cassette in lieu of metal clips to hold peristaltic tubing in place, thus reducing parts count. A stopcock and/or IV manifold at the IV cannula or patient end, if present, may be made of, or shrouded in, soft materials so that the risk of a pressure-induced injury is reduced.

The automated sheathing of the spike when an infusate container is not mounted to the cassette minimizes the risk of accidental sharps injury. The design provides tamper-resistant inaccessibility to the spike when the spike is not inserted in an infusate container, to further minimize risk of accidental sharps injury. When the infusate container entry mechanism and/or the cassette are made of plastic, the design of those elements may be compatible with constraints imposed by injection molded tool design.

Upon removal of an infusate container from the cassette, a spike sheath re-deploys to sheath the spike. The movement of the spike sheath may be used to actuate a lever arm that rotates a stopcock such that an infusate lumen in a spike assembly is closed and infusate flow is prevented. Thus, after an infusate infusion, uncontrolled free flow of residual infusate left in the peristaltic and intravenous tubing to a patient still connected to the cassette is prevented, e.g., when the cassette is removed.

A breakable fin on the cassette may be used as an indicia of the use status of the cassette. An air filter housing may be incorporated into a spike assembly to reduce parts count. A holder for the air filter media may also be incorporated in the spike assembly to further reduce parts count and manufacturing cost.

The cassette may be indexed to its mating surface by designing the cassette such that it can only mount onto its mating surface on the housing of an infusion system in a predetermined or singular orientation.

### BRIEF DESCRIPTION OF THE DRAWINGS

FIG. 1 shows a perspective view of one embodiment of an infusion system for computer assisted infusate administration according to the present invention.

FIG. 2 is a schematic showing data flow according to one embodiment of the present invention.

FIG. 3 shows a top cross-sectional view of one embodiment of the cassette fitted with an infusion system according to the present invention.

FIG. 4 shows a front cross-sectional view of an alternative embodiment of a cassette extension with an infusate container in place thereon according to the present invention.

FIG. 5 shows a perspective view of one embodiment of a redundant volume tracking system according to the present invention.

FIG. 6a shows a perspective view of one embodiment of a cassette according to the present invention.

FIG. 6b shows a perspective view of one embodiment of a cassette with infusate delivery conduit according to the present invention.

FIG. 7 is a block diagram of mechanisms for redundant volume tracking according to the present invention.

FIG. 8 is a block diagram of mechanisms for the automatic shut off of the pumping mechanism according to the present invention.

FIG. 9 is a block diagram of certain parameters used with the quality assurance modules according to the present invention.

FIG. 10 shows one embodiment of the anti-reflux valve and IV manifold connector according to the present invention.

FIG. 11 is a block diagram of one embodiment of the liquid and air flow path between various components according to the present invention.

FIG. 12 shows a front cross-sectional view of one embodiment of a cassette extension with an infusate container suspended therefrom according to the present invention.

FIG. 13 depicts a perspective view of one embodiment of a cassette with integral spike sheathing and anti-free flow features according to the present invention.

FIGS. 14a and 14b show different perspective views of a spike assembly with an integrated stopcock lever arm that interacts with a cassette according to the present invention.

FIG. 15 shows a cut-out view of one embodiment of a spike assembly attached to a cassette with a spike sheath omitted according to the present invention.

FIG. 16 shows a perspective bottom view of one embodiment of a spike sheath according to the present invention.

FIGS. 17a and 17b represent perspective cut-out views of one embodiment of an anti-free flow device on a spike assembly interacting with protuberances on a spike sheath, in sheathed and exposed positions respectively according to the present invention.

FIG. 18 shows a perspective view of a cassette and a mating surface when the two are not yet touching according to one embodiment of the present invention.

FIG. 19 shows a perspective view of interaction between a cassette and a mating surface when the two are partially engaged according to one embodiment of the present invention.

FIG. 20 shows a perspective view of interaction between a cassette and a mating surface when the two are engaged and mated according to one embodiment of the present invention.

### DETAILED DESCRIPTION OF THE INVENTION

The embodiments described below are not intended to limit the invention to the precise forms disclosed. The embodiments are chosen and described in order to explain the principles of the invention and its applications and uses, and thereby enable others skilled in the art to make and use the invention.

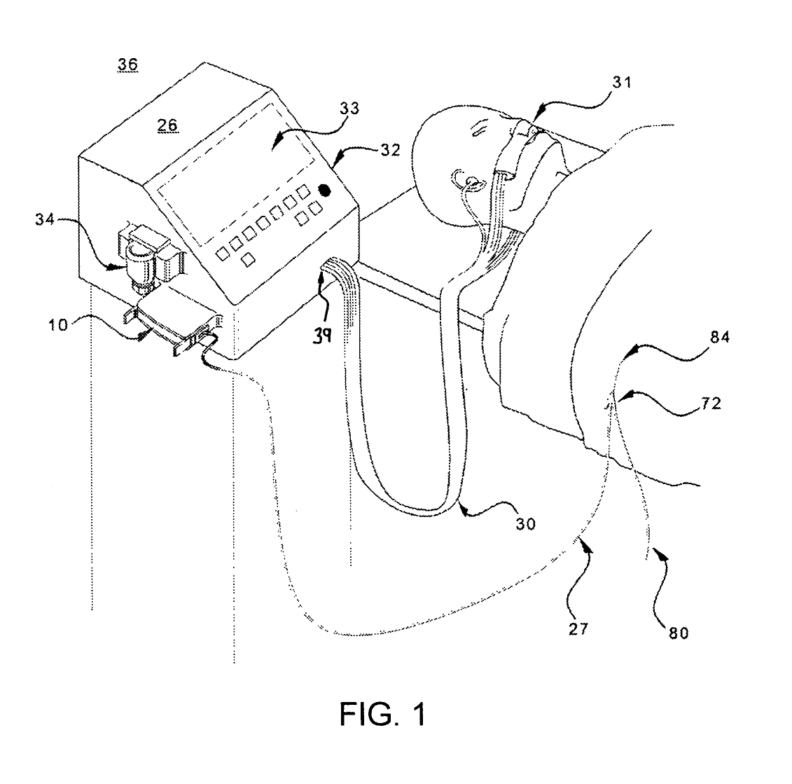

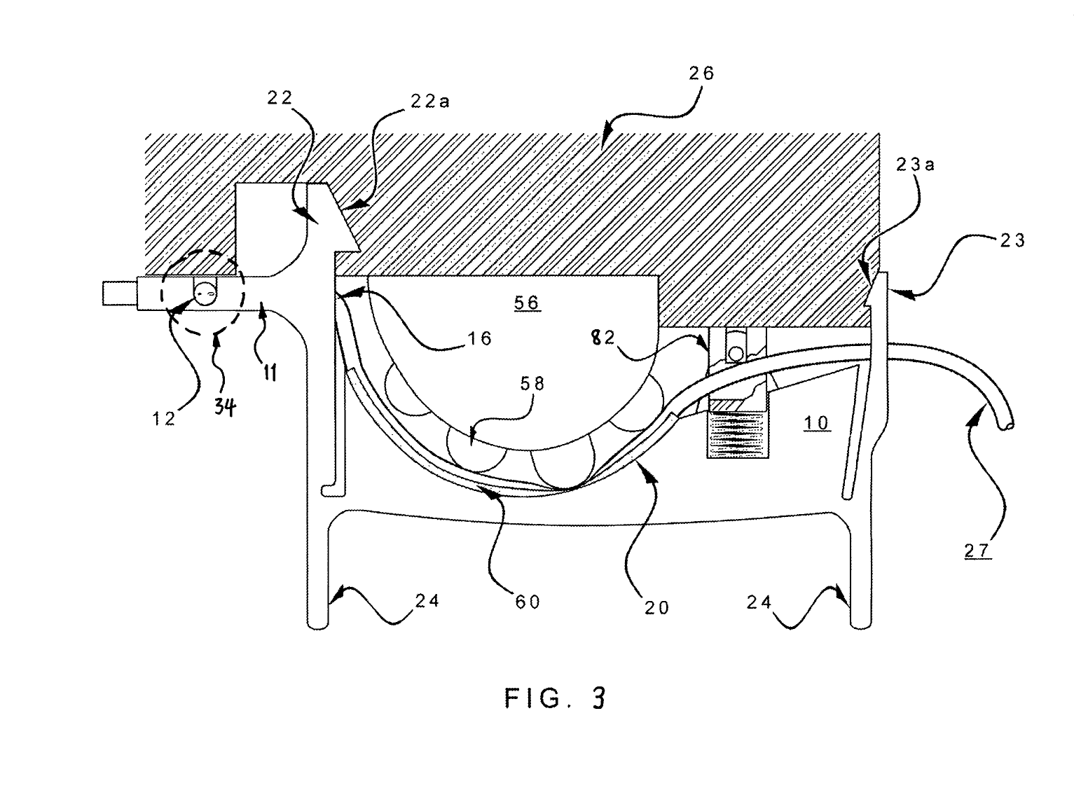

FIG. 1 shows an external view of an infusion system for computer assisted infusate administration 36 of the present invention. The system includes housing 26 for user interface 32 and pumping mechanism 56 (shown in FIG. 3), as well as ports for the attachment or insertion of, infusate container 34, detachable cassette 10 for receiving infusate container 34 and patient interface devices such as oronasal device 31 which may, for example, provide oxygen and/or capnometry or other respiratory monitoring. Infusate flows from the cassette 10 to a patient via intravenous infusion line or delivery conduit 27. Intravenous fluids, or other fluids, if used, flow to the patient via separate infusion line 80. Lines 80 and 27 merge at connector or IV manifold 72. Fluid flows from connector 72 to the patient via a vascular access device such as, among others, an IV needle, cannula or catheter 84 that is inserted in a vein of the patient. Delivery conduit 27 may be removably or permanently attached to cassette 10 and/or connector 72.

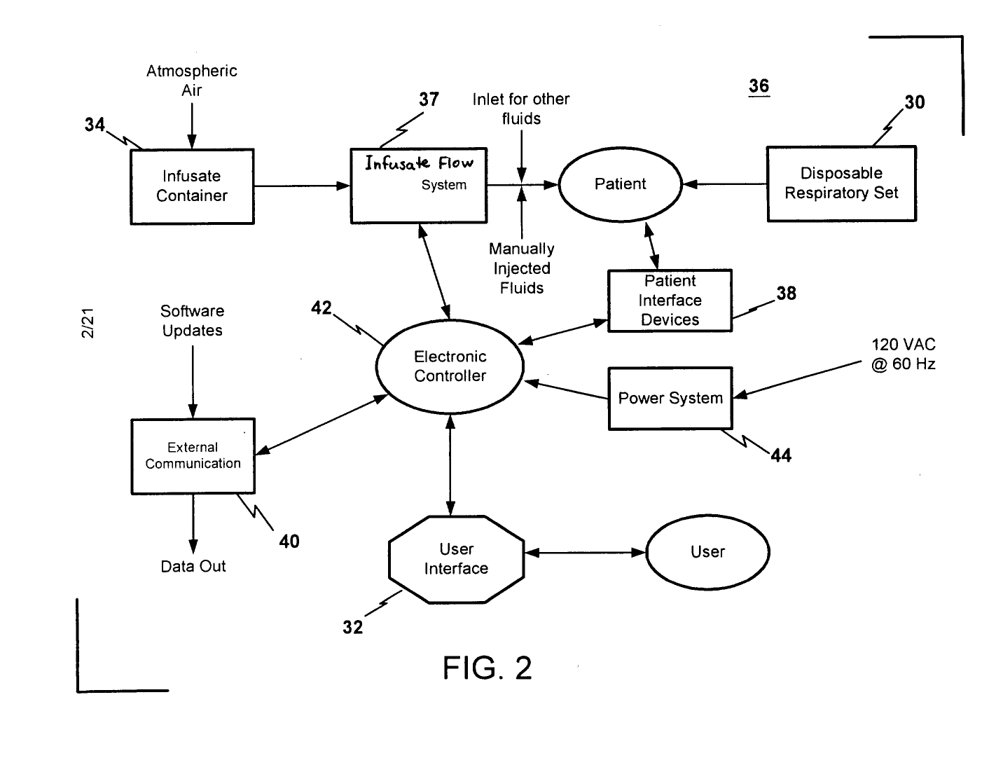

User interface 32 is connected to a microprocessor-based electronic controller or computer 42 (shown in FIG. 2) located within housing 26. The electronic controller 42 may be comprised of available programmable-type microprocessors and other chips, memory devices, and logic devices on various boards. Various user interface devices include display device 33 which may be integrated into housing 26 of infusion system 36 which displays patient and system parameters and operation status of the infusion system 36, a printer (not shown) which prints, for example, a hard copy of patient parameters indicating the patient's physiological condition and the status of the infusion system 36 and infusate flow with time stamps, and an optional remote control device (not shown) which permits a clinician to interact with the infusion system 36 from a distance. User interface 32 may include hard and soft buttons that allow the user to override an automated infusion process and manually control or interrupt the infusion as well as purge the infusion set of air or prior infusate.

FIG. 1 also shows respiratory set 30 which may be attached to infusion system 36 and which, along with oronasal device 31, may be disposable. Preferably, the respiratory set is a single-patient or single-use disposable element that is removably attachable to the infusion system 36. The infusion system 36 includes a connector port 39 within its housing 26 in which the respiratory set may be attached so that it is operably coupled with the electronic controller 42.

FIG. 2 is a schematic with data flow showing the infusion management steps performed by electronic controller 42 in an embodiment of the present invention. A user interacts with user interface 32 that is in communication with electronic controller 42 whereby the user may input certain commands or program process sequences that are then stored in memory by the electronic controller 42. The electronic controller 42 is in communication with infusion system 36 which controls flow from infusate container 34. Infusate flow system 37 may comprise pumping mechanism 56 (shown in FIG. 3), cassette 10 and delivery conduit 27, and is capable of functioning as an autonomous infusion system or can be integrated into a larger system. The electronic controller 42 monitors and regulates the infusion rate based on input from the user, from control software that may incorporate drug state models, and/or from data collected from patient interface devices 38. The various patient interface devices 38 can include one or more patient health monitors (not shown) that monitor a patient's physiological condition, such as a pulse oximeter, capnometer, blood pressure monitors, EEG, EKG, responsiveness monitor, airway pressure monitors, among others.

FIG. 2 also shows respiratory set 30 (which may be disposable and include oronasal device 31) which is connected to a patient; a power system 44 which provides power to electronic controller 42; and external communication device 40 which may be a printer and in communication with electronic controller 42 and which accept software updates and output data.

FIG. 3 shows a cassette 10 for the transfer of infusate from infusate container 34 (which may be sealed) to a patient. The cassette 10 provides a mechanical platform for anchoring infusate container 34 to the housing 26 and assures that the infusate container 34 remains at a fixed head height with respect to pumping mechanism 56. The cassette 10 also assures that delivery conduit 27 is positioned and oriented properly with respect to pumping mechanism 56. The cassette includes an extension 11 for receiving the infusate container 34 and maintaining the container's position during the infusion process.

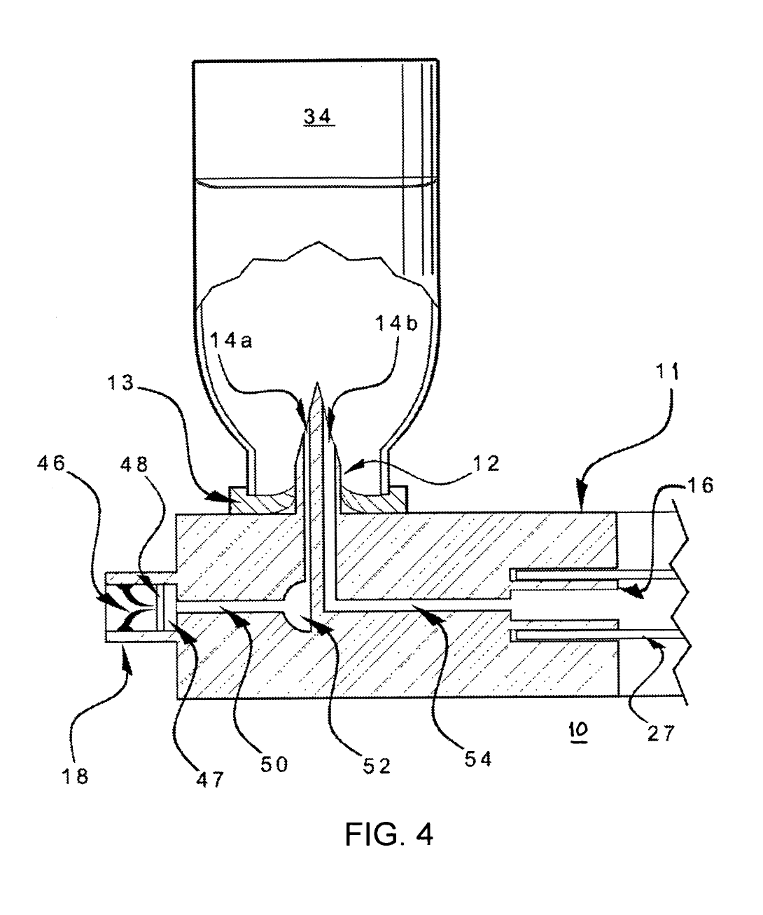

In a particular embodiment of the invention, the cassette 10 receives a single infusate container 34 for each infusion process. At the conclusion of the infusion process or upon the near-depletion of the container 34, the container 34 is removed and the cassette 10 may receive a new infusate container 34 for an extension of a prior infusion process. The delivery conduit 27 may be purged of any air and/or infusate from the prior infusion process. In an alternative embodiment, the cassette 10 may receive more than one infusate container 34 at a time. The cassette 10 may have multiple flow lumens (e.g., such as those shown in FIG. 4 at 54) to channel the infusate flow from each of the separate infusate containers into a single infusion system within the cassette 10 or infusion system 36. A mechanism may be provided to restrict the infusate flow created by the pumping mechanism 56 so that infusate flows from one infusate container 34 at a time for a sequential sequence or from more than one infusate container 34 at a time according to pre-determined proportions. Pumping infusate from multiple containers simultaneously allows an extended infusion run without halting for a purge sequence. Pumping infusate from multiple containers in concert allows separate and segregated sources of infusate to be used concurrently for a single infusion run. In a further alternative embodiment, multiple containers of the same infusate are provided with a single cassette 10 such that one container can be removed while infusate is flowing from another. Such an embodiment allows for an extended infusion process without halting for a purge sequence.

Still referring to FIG. 3, the extension 11 may include an attached infusate flow activation device 12 (which as further described below may be a spike or other sharp having internal lumens) for initiating the transfer of the infusate from the infusate container 34 to delivery conduit 27. At the start of the infusion process, the infusate container 34 is placed onto the activation device 12.

FIG. 3 also shows an opening 16 in the cassette where infusate flow lumen 54 (as shown in FIG. 4) within the extension 11 terminates. One end of pressure plate 20 is located near opening 16. The pressure plate is rigid enough to provide a platform against which pump fingers 58 may operate. A rigid pressure plate also allows cassette 10 to be easily fitted onto its mating surface on housing 26 with a one-step snap on motion. In one embodiment of the present invention, the pressure plate 20 has a concave curve that bowls away from the opening in order to accept the curved face of pumping mechanism 56. Alternatively, a flat pressure plate 20 and a flat face of a pumping mechanism 56 as well as other pressure plate profiles may also be used with the present invention.

FIG. 3 further shows an infusate delivery conduit 27 that is provided with the cassette 10 at opening 16. Delivery conduit 27 is inserted over the male port in opening 16 to create an air-tight connection with the flow channel created by infusate flow lumen 54 (shown in FIG. 4). Delivery conduit 27 is positioned along the pressure plate 20 such that pumping mechanism 56 may act on it to move the infusate through the conduit, away from the infusate container 34, and to the patient. The delivery conduit, which may be tubing, may be fixed in position along the pressure plate 20. A structure to hold the delivery conduit 27 abutted against the pressure plate 20 should not interfere with the action of peristaltic pump fingers 58. Several embodiments of such structure are contemplated for affixing the delivery conduit 27 to the pressure plate 20. For example, the conduit 27 may be ultrasonically welded or glued to the pressure plate 20 or it may be fitted within foam strip guides 60, which are themselves fixed to the pressure plate 20. The foam strip guides 60 by virtue of being compressible and collapsible do not interfere with the accuracy of the pumping mechanism 56 or the operation of pump fingers 58. Alternatively, pieces of plastic tubing similar to delivery conduit 27 could be placed on pressure plate 20 above and below delivery conduit 27 such that they hold delivery conduit 27 securely against pressure plate 20 and collapse when squeezed by pump fingers 58. At least a portion of delivery conduit 27 may be transparent so that the user can observe the infusate flow through the conduit and visually check for, among other things, entrained air or particulates in, or denaturation, separation or emulsification of, the infusate.

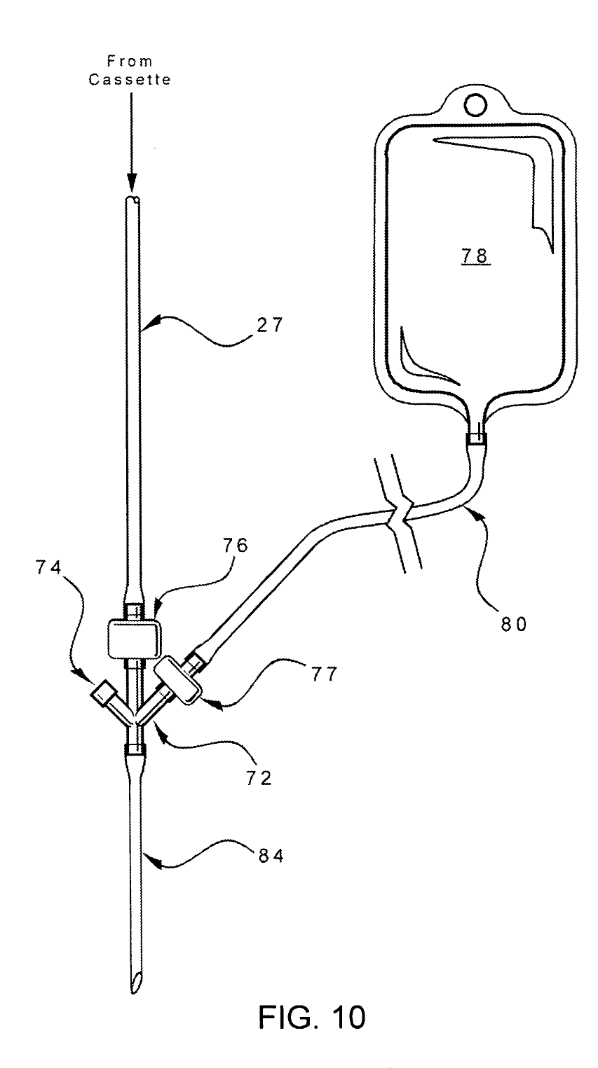

The pumping mechanism 56 may be a peristaltic pump with at least three movable fingers 58 which act upon delivery conduit 27 and against pressure plate 20 so as to create a pressure gradient within the delivery conduit. The pressure gradient causes the infusate to flow from the infusate container 34 into the bore 14b (shown in FIG. 4) within the spike, then into the infusate flow lumen 54 (shown in FIG. 4) within the cassette extension 11, then into the delivery conduit 27, and then through manifold connector 72 (shown in FIG. 1 and FIG. 10) and into vascular access device 84 inserted in a vein of the patient. Because the pump fingers 58 are external to the delivery conduit 27 and the entire infusion system tubing, the pumping mechanism 56 may be able to operate even if air is in the active pumping section of the delivery conduit 27. The pumping mechanism 56 may be controlled manually or by the electronic controller 42 (shown in FIG. 2) of an infusion system 36 and may be set at a given flow rate or at a specified gradient, rate of change over time or time profile of infusate flow rates.

FIG. 3 also shows spring-loaded clamp or pinch valve 82 which acts as a free flow prevention device to halt the unchecked or free flow of infusate to the patient by gravity when the cassette 10 removed from contact with the pumping mechanism 56. Clamp 82 pinches a portion of conduit 27 closed when it is not being kept open, e.g. by contact with housing 26, pumping mechanism 56 or infusion system 36.

As shown in FIG. 3, the cassette 10 may also include one or more extensions such as snap locks 22 and 23 which provide mechanical attachment to housing 26 such that the cassette 10 may be fixed in place relative to its mating surface and pumping mechanism 56. In a particular embodiment, these extensions fit into slots 22a and 23a on the mating surface of housing 26 allowing for a snap-on single motion attachment of the cassette 10. The cassette 10 may also include finger grips 24 for gripping cassette 10 and guiding it into its designated place within the housing 26. When finger grips 24 are squeezed together, snap locks 22 and 23 are spread apart allowing the cassette to be placed into slots 22a and 23a.

FIG. 4 shows a particular embodiment of infusate flow activation device 12 in which it is an upright spike for piercing a resealable stopper 13 of an inverted infusate container 34. The spike 12 includes bore 14b which creates an air-tight opening in the container 34 out of which the infusate may flow. Extension 11 of cassette 10 contains infusate flow lumen 54 provided between bore 14b and infusate flow opening 16 in the cassette 10. One end of delivery conduit 27 may connect to opening 16 while the other end may be attached to connector 72 (shown in FIG. 1 and FIG. 10). Extension 11 may also contain an air flow lumen 50 between another bore 14a in spike 12 and an opening to atmosphere through inlet 18.

Infusate container 34 is generally inert to the infusate and impermeable to atmospheric contaminants. The container 34 is capable of protecting the infusate from outside contamination prior to and during the infusion process. Preferably, infusate container 34 is a rigid vial of invariable volume, though a flexible container such as a collapsible IV bag is also contemplated for use with the present invention. The infusate container 34 may have at least one transparent portion to allow visual assessment of the infusate's condition and volume. The infusate container 34 may also include a built-in gripping device such as a molded tab (not shown) by which a user can hold and transport the container without contaminating its surface. Preferably, self-sealing stoppers 13 are used with infusate containers that are to be removed from the cassette after use. Self-sealing stoppers provide air-tight piercing, prevent infusate spillage, and help to prevent the infusate from being compromised due to evaporation or contamination.

Still referring to FIG. 4, extension 11 may include a one-way or pressure relief valve 46 through which atmospheric air is introduced into infusate container 34 in order to prevent excessive vacuum (that might interfere with infusion) from developing above the infusate's meniscus as the infusate flows out of the container. Air flow lumen 50 is provided between one-way valve 46 and bore 14a in spike 12. Because in the embodiment depicted in FIG. 4 infusate can flow by gravity along air flow lumen 50 to the atmosphere, certain embodiments are contemplated to prevent infusate from leaking out of the air flow lumen 50 while still allowing air to bleed inside the infusate container 34 to prevent formation of an excessive vacuum. In one of these embodiments, the mechanism to prevent infusate spillage from bore 14a is a one way valve 46. One-way valve 46 only allows atmospheric air into air flow lumen 50 and does not allow any infusate which has leaked through bore 14a to escape the cassette 10.

In a further infusate leakage prevention embodiment, a hydrophobic filter 47 is provided with air flow lumen 50 in the extension 11. The hydrophobic filter 47 prevents any infusate which has leaked into air flow lumen 50 of the spike 12 from flowing out of air inlet 18 of the cassette 10.

In a further infusate leakage prevention embodiment, bore 14b is a wide bore and bore 14a is a narrow bore. Narrow bore 14a is in communication with air flow lumen 50 in the extension 11 while wide bore 14b is in communication with infusate flow lumen 54 of the extension 11. The difference in capillary action caused by the different bore sizes causes the liquid infusate in the infusate container 34 to tend to flow through wide bore 14b and into infusate lumen 54 only. Capillary action hinders the flow of infusate into the narrower air flow lumen. In an additional infusate leakage prevention embodiment, air flow lumen 50 contains a half-moon-shaped well 52 so as to restrict the flow of any infusate that does leak into air flow lumen 50 from making it to air inlet 18.

An air filter 48 may be provided with air inlet 18 to prevent particulates in atmospheric air from entering air flow lumen 50 inside the extension 11 and inside infusate container 34. Air filter 48 may be capable of screening out microbial matter including bacterial and viral particles.

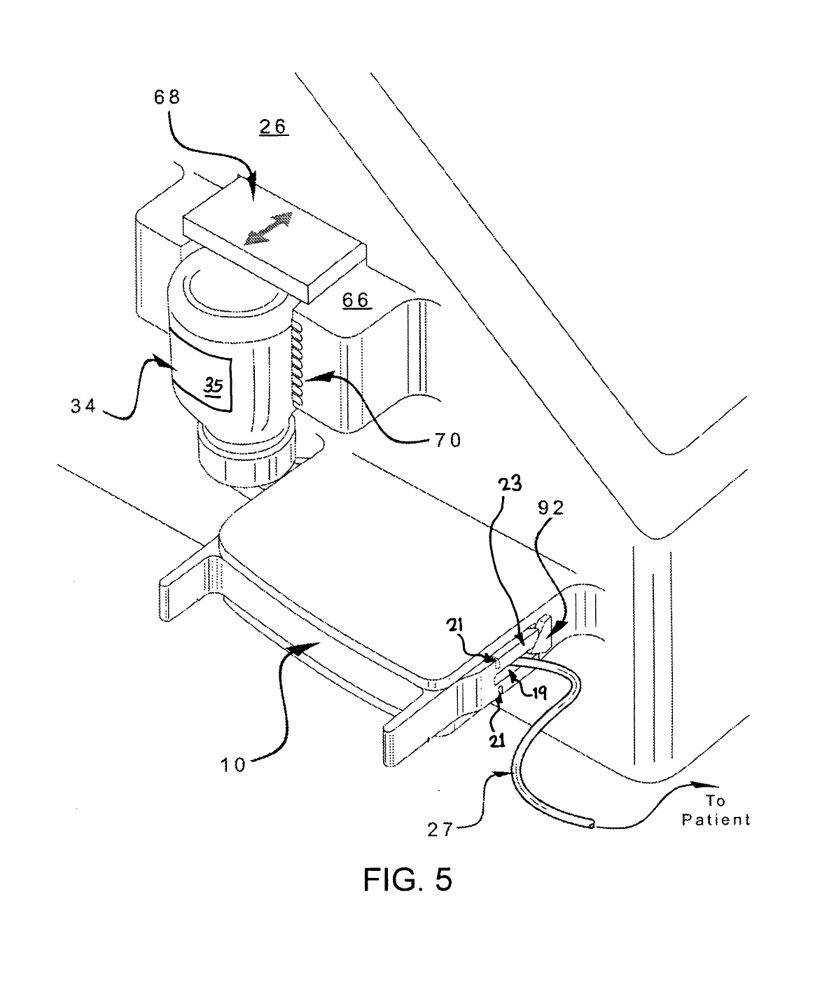

In an alternative embodiment of the present invention, the infusate container 34 may include a pre-attached spike 12 and the container-spike set may be inserted as a unit onto the extension 11. In a further alternative embodiment, the cassette 10 with extension 11 may include a pre-positioned infusate container 34 with an intact, i.e., not punctured, seal 13 which may be spiked (e.g., manually) immediately prior to activation of the infusion system. With such embodiments, the entire cassette-infusate container assembly may be fixed to the infusion system as a single unit, activated, and used and then may be subsequently removed from the housing 26 (shown in FIGS. 1 and 5) and disposed of as a single unit.

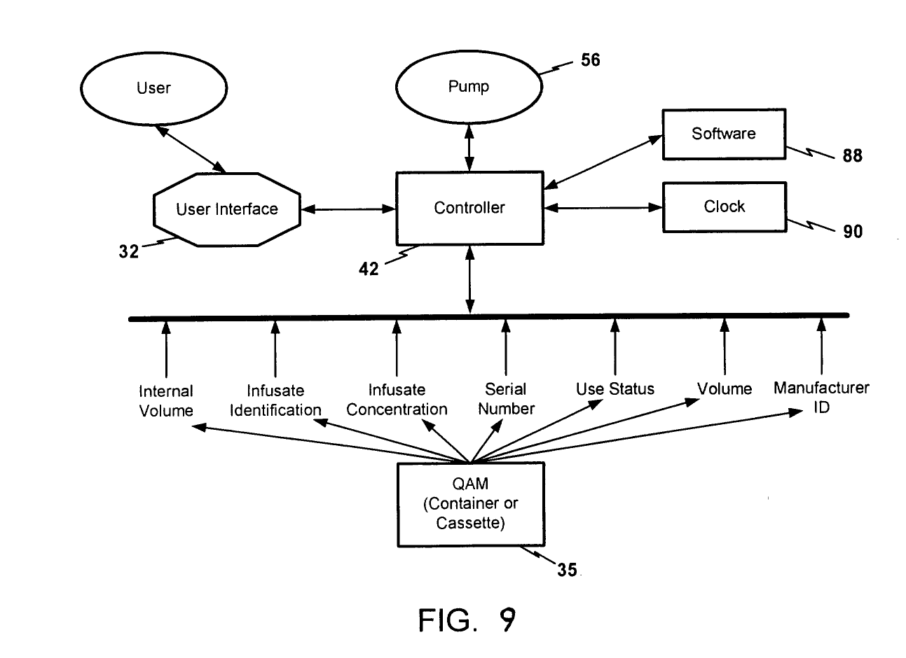

FIG. 5 shows an array 70 of photo-emitter cells and photo-detector cells, which may be an element used in an alternative redundant volume tracking method according to the present invention. Such an array may be provided with the cassette 10 or as part of housing 26. Each photo-emitter cell emits light directed at the infusate container 34. The difference in reflection of the emitted light depending on whether it impinges on air or infusate, especially milky infusates like propofol, is used to track the meniscus. Emitted light which reflects back from the liquid inside of the container is detected by a photo-detector cell. The detector cells are capable of receiving reflected light from the infusate and are arranged in a pattern, such as a column, whereby if a particular detector cell receives a certain amount of reflected light, then it is below the meniscus of the infusate and whereby if the particular detector cell receives a different amount of reflected light, then it is above the meniscus of the infusate. The photo-detector cells can measure reflected light when they are on the same side of the infusate container 34 as the emitters or transmitted light when the detectors are on the opposite side of the emitters. Each cell of the array is in communication with an electronic controller 42 (shown in FIG. 2) and the controller 42 determines where the meniscus is within the infusate container 34 by identifying the region where there is a sharp transition in reflected or transmitted light. Meniscus tracking allows independent calculation of how much infusate remains in the infusate container 34 based on the initial volume of the infusate in the container 34. The initial volume of the infusate in the container 34 may be encoded as a volume value and/or a particular meniscus level corresponding to a full container and/or a container characteristic such as cross-sectional area for a given container size on a quality assurance module (“QAM”) 35 located on the given container 34. QAM 35 is described in more detail below with regard to FIG. 9. A mechanism is provided to read the information on the QAM 35 and transmit among others data related to the initial volume to the electronic controller 42. The photo emitter/detector pairs of array 70 may be staggered in two or more separate arrays to provide more spatial resolution.

FIG. 5 also shows an alternative embodiment of a free flow prevention device that may be provided with the present invention. Snap lock 23 of cassette 10 contains a slit 19 through which delivery conduit 27 may be placed. Cut-outs 21 are provided at each side of the slit 19 to allow the slit 19 to be forced wide apart such that when the cassette 10 is placed into proper position on to housing 26 a spreader piece 92 located on housing 26 spreads the fingers of snap lock 23 allowing the unrestricted flow of infusate through delivery conduit 27.

Still referring to FIG. 5, housing 26 may include mechanical receptacle 66 for receiving and supporting the infusate container 34 as the infusate is drawn out of the container 34. The receptacle 66 may be a particular size capable of receiving a particularly sized infusate container 34 or it may be structured so as to receive containers of variable sizes.

FIG. 5 also shows an embodiment of an infusate container removal lockout mechanism 68 that may be provided with the housing 26 to prevent the removal of container 34 while the pumping mechanism 56 (shown in FIG. 3) is running. When in a locked position, mechanism 68 slides out of housing 26 and mechanically prevents removal of the infusate container 34 from the cassette 10. Mechanism 68 may be in communication with the infusion system electronic controller 42, which will only signal the pumping mechanism 56 that it may run when the lockout mechanism 68 is in a locked position. When mechanism 68 is in an unlocked position and retracted, the infusate container 34 may be physically removed from the cassette 10 and the electronic controller 42 will signal the pumping mechanism 56 to halt the infusate flow. Once a new infusate container 34 is inserted on the cassette 10 and the lockout mechanism 68 is returned to a locked position, the electronic controller 42 will again signal the pumping mechanism 56 that it may run. If the electrical power system 44 (shown in FIG. 2) or software to controller 42 fails, mechanism 68 can be manually pushed back into housing 26 to allow removal of container 34. This feature of the present invention may remove the need for a purging sequence each time an infusate container 34 is removed and replaced by another container containing an infusate with the same identity and concentration as the infusate of the prior container.

In certain embodiments, the infusate container lockout mechanism 68 may be implemented by software running on controller 42. A request for removal of the infusate container 34 is received by the software. The software checks whether infusion is ongoing and may decide based on the context and prevailing conditions whether to stop infusion to allow infusate container removal or allow infusion to continue and prevent infusate container removal, and depending on the decision may send an appropriate command to an actuator that can prevent infusate container removal to either allow or prevent manual removal of the infusate container 34.

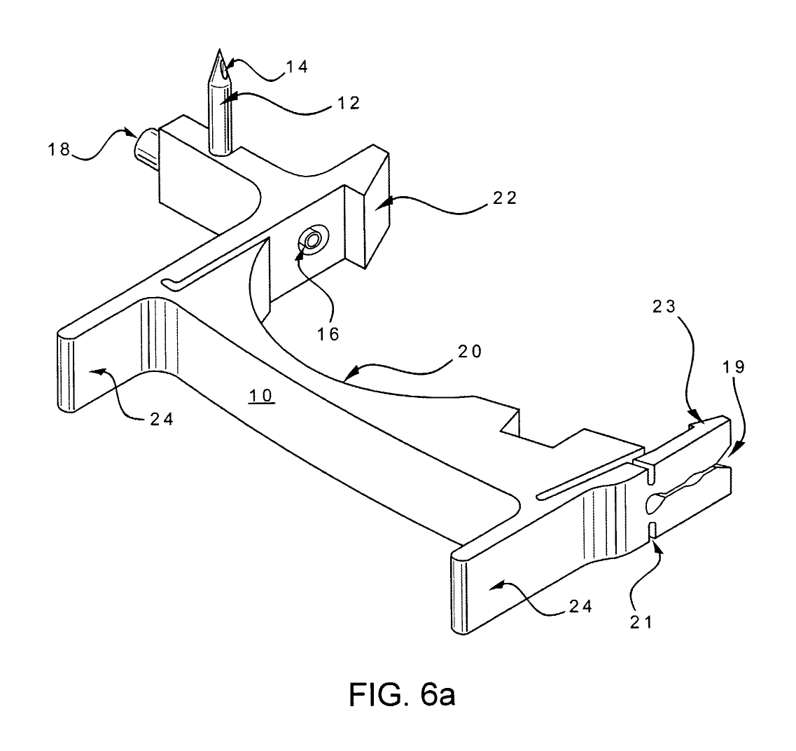

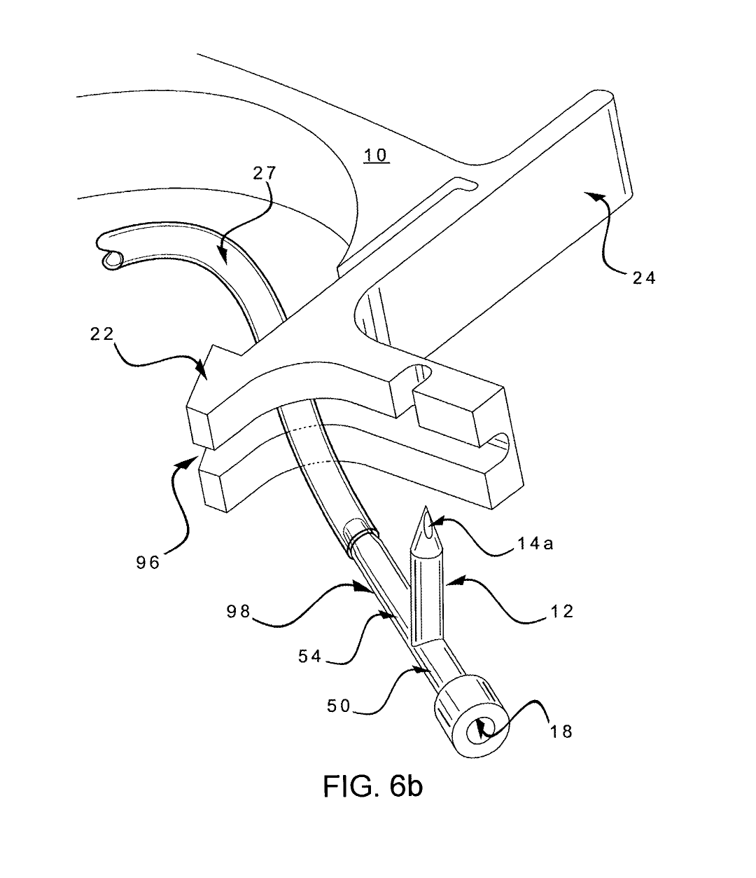

FIG. 6a shows a further perspective of cassette 10 where the cassette is not attached to housing 26. Each of finger grips 24, pressure plate 20, opening 16, spike 12 having bore 14, air inlet 18, snap locks 22 and 23, slit 19, and cut-outs 21 (all described in detail above) are shown.

FIG. 6b shows an alternative embodiment of cassette 10 in which spike 12 is attached to delivery conduit 27 and can be removed from cassette 10 so that the cassette itself might be reusable with a new spike assembly 98. Spike assembly 98 fits into conduit 27, which fits into slot 96 of the cassette 10. When the cassette 10 is placed against housing 26 (not shown), the housing 26 helps to keep spike set 98 securely held within slot 96.

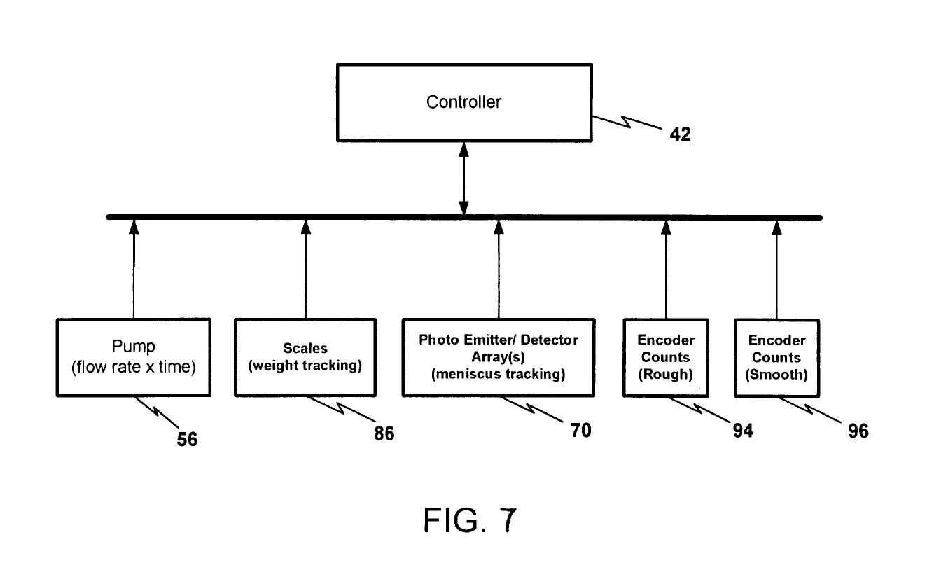

FIG. 7 illustrates various mechanisms for tracking the volume of infusate pumped out of the infusate container 34 during the infusion process. Methods for volume tracking provide redundancy to the volume calculated by the infusion system electronic controller 42 from the cycles of the pumping mechanism and the duration of the infusion so that the accuracy of the pumping mechanism's 56 flow rate may be verified and compensated for. This redundancy helps ensures a dependable and accurate flow rate of infusate into the patient.

One such mechanism for redundant volume tracking utilizes scales 86 which measure the weight of the infusate container 34 as it is in contact with the infusate flow activation device 12. The scales 86 may be provided with the cassette 10 or as part of the infusion system 36. The scales 86 are in communication with the electronic controller 42 which receives either continuous or periodic data on the weight of the infusate container 34 and its remaining contents. As infusate flows out of the container 34, the weight decreases and the electronic controller 42 calculates the corresponding decrease in infusate volume from a preprogrammed set of infusate density data. By monitoring the change in volume over a given amount of time, the average flow rate over that given amount of time may also be calculated.

Another volume tracking mechanism is the photo emitter/detector array 70 for meniscus tracking described above with reference to FIG. 5.

Further volume tracking may be provided by tracking internal encoder counts 94 and 96 of the pumping mechanism 56. Because most pumps use a motor to drive the pumping mechanism, there is typically a set volume of infusate delivered with each revolution or cycle of the pump's motor. If an encoder mechanism, such as a set of optical emitter/detector cells capable of detecting the passage of slots in the pump's cam, is provided with the pump, each revolution of the pump's motor can be detected. The electronic controller 42 can multiply the number of revolutions per minute of the pump's motor by the volume of infusate delivered per revolution to derive the infusion rate in volume per minute. The controller 42 can then integrate flow rate over time to calculate the total volume infused over time and derive average flow rate too.

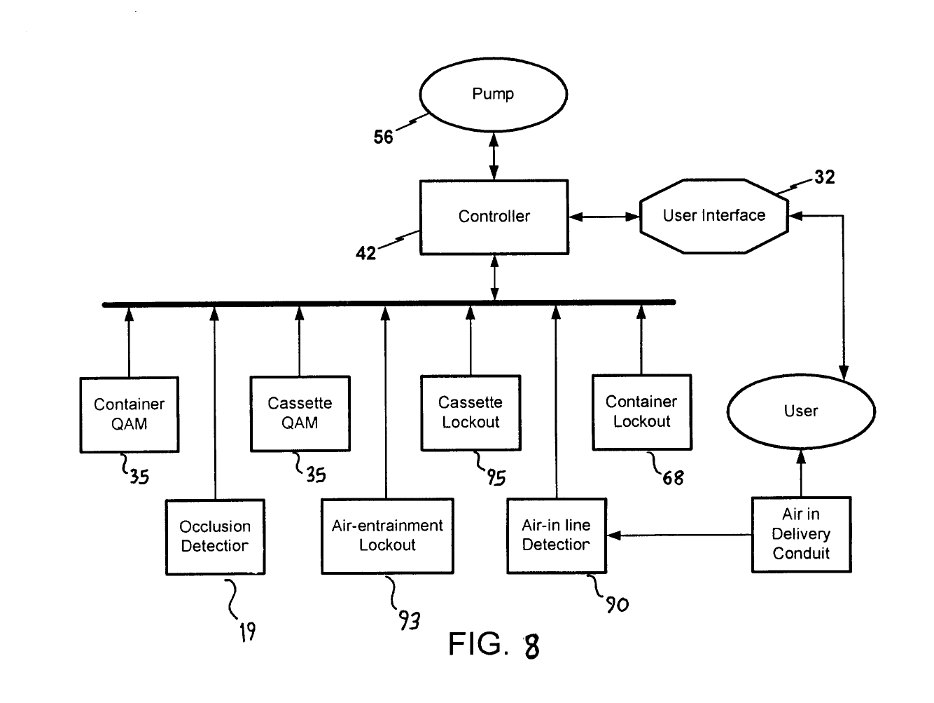

FIG. 8 shows various optional methods for alerting the electronic controller 42 of reason to shut off the pumping mechanism 56. These methods help to prevent air from being pumped into a patient's blood circulation and help to prevent an incorrect (e.g., expired, previously used, or unrecognized) infusate or an incorrect dose from being administered to a patient.

In one of these methods, the user manually signals for a pump shut down if air is observed traveling towards the patient. The user interacts with a user interface 32 (shown in FIG. 1) which is in communication with the electronic controller 42. An air-in-line detector 90 may also be provided within the infusion system 36 to sense air bubbles within the infusate. The air-in-line detector 90 is in communication with the electronic controller 42. The electronic controller 42 may be programmed to send a signal to the pumping mechanism 56 to terminate the flow rate upon notice of a signal from the air-in-line detector 90. The conduit or PVC tubing 27 may then be purged of air.

In another of these methods, at least one occlusion detector 91 is provided with the cassette 10 or with the infusion system 36 to sense via associated pressure changes whether a kink or obstruction to flow is present in the delivery conduit 27. The occlusion detector 91 is in communication with the electronic controller 42 and sends a signal to the controller 42 when such an obstruction is detected. The controller 42 may be programmed to send a signal to the pumping mechanism 56 to terminate the flow rate upon notice of a signal from the occlusion detector 91.

In yet another of these methods, an air-entrainment lockout mechanism 93 is provided with the cassette 10 or with the infusion system 36. An air-entrainment lockout mechanism 93 is triggered by the removal of an infusate container 34 from the cassette 10 while the pumping mechanism 56 is running. Once triggered, the air-entrainment lockout mechanism 93 halts the flow of infusate within the cassette 10.

An example of an air-entrainment lockout mechanism 93 is a micro-switch located on or near the infusate flow activation device 12. When the infusate container 34 is removed from the activation device 12 it triggers the micro-switch to send a signal to the electronic controller 42. The micro-switch may be a spring-loaded button that is depressed as long as the infusate container 34 is on the activation device 12 and is released when the container 34 is removed, it may be a spring-loaded button positioned in such a location as to be depressed by the surface of the infusate container 34 as the container is removed, or it may be an electronic sensor such as an optical, electromagnetic, inductive or capacitive sensor that registers when the infusate container 34 is removed.

An example of an infusate container removal lockout mechanism 68 and pump 56 management with respect to such a lockout mechanism is described above with respect to FIG. 5.

Still referring to FIG. 8, in a further particular embodiment, a cassette removal lockout mechanism 95 may be provided with the infusion system 36 to prevent the removal of the cassette 10 while the pumping mechanism 56 is running. When in a locked position, the mechanism 95 mechanically fastens the cassette 10 to housing 26. The mechanism 95 may be in communication with the electronic controller 42, which will only signal the pumping mechanism 56 that it may run when the lockout mechanism 95 is in a locked position. When in an unlocked position, the cassette 10 may be physically removed from the housing 26 and the electronic controller 42 will signal the pumping mechanism 56 to halt the infusate flow. Once a new cassette 10 is fitted within the housing 26 and the lockout mechanism 95 is returned to a locked position, the electronic controller 42 will again signal the pumping mechanism 56 that it may run. The mechanical cassette lockout mechanism 95 may be readily implemented by manually operated or motorized brackets, locks, twist locks, cams, levers, or any mechanical part that, when extended, physically prevents removal of the cassette. Sensors such as, among others, microswitches, proximity sensors, capacitive, magnetic, Hall effect, optical and inductive sensors may monitor the position of the manually operated or motorized cassette lockout mechanisms 95 and may communicate this data to controller 42.

The cassette lockout functionality may also be implemented via software (which can be run on electronic controller 42) whereby the software receives a request or indication of a request to allow removal of the cassette 10, then checks the prevailing conditions (e.g., among others, whether infusate is being infused, whether an end of case has been signaled, whether the cassette 10 has been flagged as non-QAM compliant), and then allows the cassette 10 to be removed (manually or automatically) if it is safe to do so. In normal operation of the software implementation, the cassette 10 may only be removed via a request to the control software. In the case where the cassette 10 is manually removed, the software may control a motorized lockout mechanism 95 that can not be manually activated in normal operation. In an emergency, the user is allowed to override the software and remove the cassette 10 after at least one warning message that the user has to acknowledge. Different ways to combine mechanical and software cassette lockout features into hybrid designs will be known to one skilled in the art.

In yet another of the optional methods for alerting the electronic controller 42 to shut off the pumping mechanism 56, various QAMs 35 which can be attached to the cassettes 10 and containers 34 are contemplated which store information to be communicated to the electronic controller 42. If a parameter recorded on a QAM 35 is out of a preprogrammed range stored in memory by the electronic controller 42, then the controller 42 may send a signal to the pumping mechanism 56 to terminate or not initiate infusion.

FIG. 9 is a block diagram of certain parameters that the infusate container QAM 35 and cassette QAM 35 may store. Tags on the infusate container 34 or cassette 10 may store such parameters as the identity, concentration, initial volume or meniscus height of an infusate, characteristic dimensions or volumes of infusate containers, container identification, internal volume of the infusion set and cassette 10, density of the infusate, serial number, batch number, expiration date, address such as a Universal Resource Locator (URL) and manufacturer identification in a barcode or RFID integrated circuit for example. Examples of such tags and QAMs and their uses with integrated infusion systems are described in U.S. patent application Ser. No. 10/151,255 filed May 21, 2002 and application Ser. No. 60/324,043 filed Sep. 24, 2001, each of which is incorporated herein by reference.

The electronic controller 42 receives the parameter data from the QAMs 35 and processes it to determine the initial conditions of the infusion setup. The controller 42 may use infusate identity data encoded on a QAM 35 to authenticate product source and quality and ensure that the particular infusate to be infused is the infusate intended for the current patient. When combined with a hospital information system that may store such data as, among others, the history and physical record and known allergies of a patient, the inadvertent administration of infusate contra-indicated for the patient may be flagged and averted.

The electronic controller 42 may also use the infusate identity information encoded on infusate container or cassette QAMs 35 to determine when cross-contamination may occur. The controller 42 may store in memory the identity and concentration of a prior infusate in use and the identity and concentration of a subsequent infusate to be used with the same cassette 10 and infusion system 36. If the stored identity or concentration of the subsequent infusate is different from the prior infusate, the electronic controller 42 may automatically initiate a purging sequence to clear any residual infusate from the prior infusion sequence from the system 36.

In a particular embodiment, the electronic controller 42 uses data from the QAMs 35 to coordinate an automated purging or priming sequence. A QAM 35 on the cassette 10 may store the internal volume between the infusate container 34 and vascular access device such as, among others, the internal volume of the infusate flow lumens in the cassette 10, delivery conduit 27 and IV manifold 72 (FIG. 10). The electronic controller 42 records these internal volumes or their sum from the QAMs 35 and signals the pumping mechanism 56 to cause a volume of infusate in excess of the sum of the internal volumes to flow through the infusion set to clear any air or prior infusate remaining in the lines. An automated purging sequence allows for the precise control of the volume of infusate pumped through the IV system during a purge sequence so that just enough volume of infusate is pumped to assure that the infusion set is free of air or prior infusate. Such a purging or priming sequence performed manually may result in a greater than necessary volume of infusate being pumped out of the infusion system resulting in wasted infusate and time. The automated aspect of the purging sequence automatically reminds a user to purge or prime an IV set preventing a hazard that may result from an error of omission; it also provides an “initiate and forget” benefit whereby a user can move on to other tasks while a purge is occurring, after initiating a purge sequence.

Preferably, the electronic controller 42 references a clock 90 (FIG. 9) to establish the start time and duration of each infusion run. The controller 42 may also use the clock 90 to determine when pre-programmed events such as pump flow rate or infusate container changes should occur. The controller 42 may also use the clock 90 and the infusion rate over a given time period to determine how much infusate is left in the container 34 so as to shut off the pump 56 when the volume of infusate remaining in the container 34 is low and alert the user.

FIG. 10 shows an anti-reflux valve 77 on connector 72 connecting delivery conduit 27 with tubing 80 from the IV solution, or other fluid, container 78 and vascular access device 84. Anti-reflux valve 77 prevents the retrograde flow of infusate from tubing 27 into IV tubing 80.

A check valve 76 that is part of connector 72 prevents back flow of fluid from tubing 80 up infusate line 27. Check valve 76 can also operate as an automated free flow prevention device by deliberately increasing its cracking or opening pressure such that it is higher than the highest hydrostatic pressure generated by a spiked and full infusate container 34 with conduit 27 fully extended to its highest possible elevation. The design thus requires pumping mechanism 56 to generate more pressure than the opening pressure of valve 76 for infusate to flow to the patient. If the pump mechanism 56 (shown in FIG. 3) is not in contact with conduit 27 and pressure plate 20, when cassette 10 is removed from housing 26 or infusion system 36 for example, infusate flow will stop because the highest hydrostatic head that can be generated will be lower than the cracking pressure of valve 76.

In an alternative embodiment, connector 72 may also include a stopcock or resealable injection port 74 capable of accepting a syringe tip or needle and allowing the direct injection of infusate or fluids therefrom. A vascular access device 84 may be inserted into the patient's vein. Preferably, the vascular access device is a single-patient or single-use disposable element that is removably attachable to the connector 72.

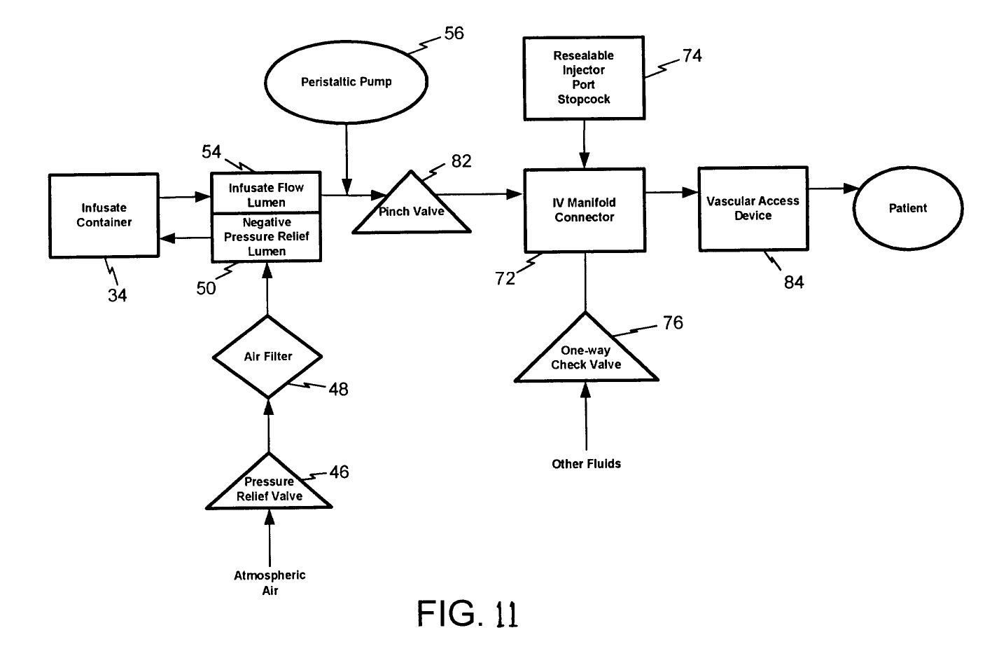

FIG. 11 shows a block diagram of an embodiment of the present invention and depicts the infusate and atmospheric air flow pathways through the elements of FIGS. 3 and 10 described above. Pinch valve 82 is open when the cassette 10 is snapped onto housing 26 or infusion system 36. As soon as cassette 10 is snapped off, the spring in pinch valve 82 extends and closes off IV line 27. The purpose of pinch valve 82 is to prevent free flow of infusate by gravity to the patient, when flow through conduit 27 is no longer being controlled by pumping mechanism 56 because conduit 27 is no longer in contact with it.

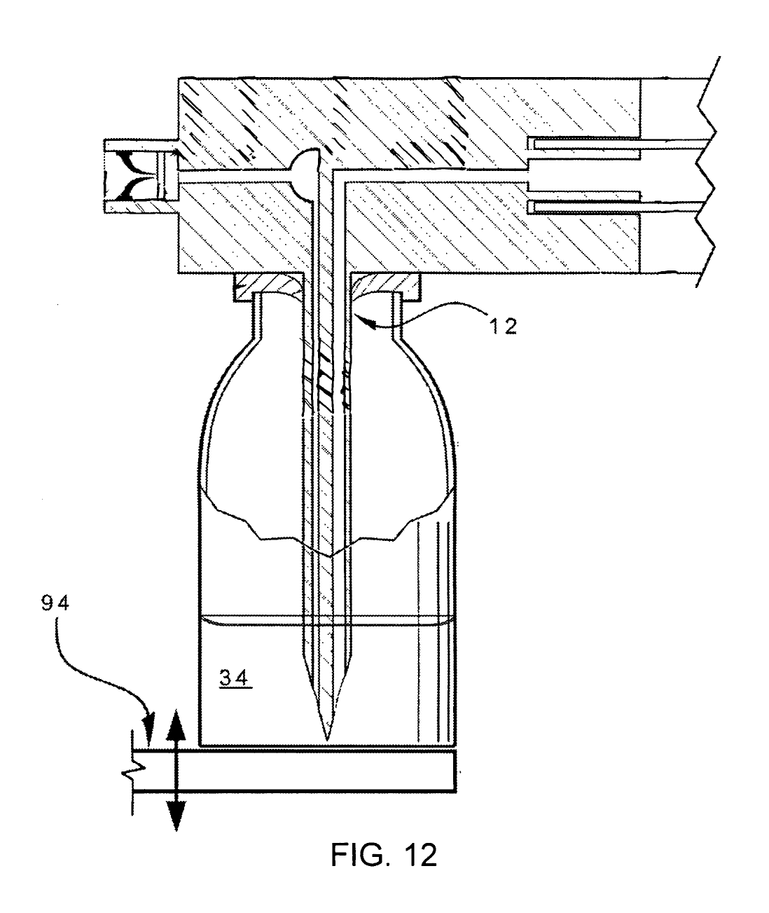

FIG. 12 shows an alternative embodiment in which infusate flow activation device 12 allows transfer of infusate from an upright infusate container. An elevator 94 is used to raise upright infusate container 34 into communication with the activation device 12. Preferably in this embodiment, an inverted spike is used as the activation device 12. If the infusate container 34 is placed upright as in FIG. 12, the possibility of the liquid contents flowing out by gravity via an air venting lumen is eliminated.

Particular alternative embodiments of the cassette 10, an anti-free flow device, an air entrainment lockout mechanism, means of securing tubing to the cassette with a minimum of individual parts, quality assurance tags, as well as a means for sheathing the infusate flow activation device (or spike) 12 when it is not in use, and stopcocks made of, or shrouded in, soft materials will now be described.

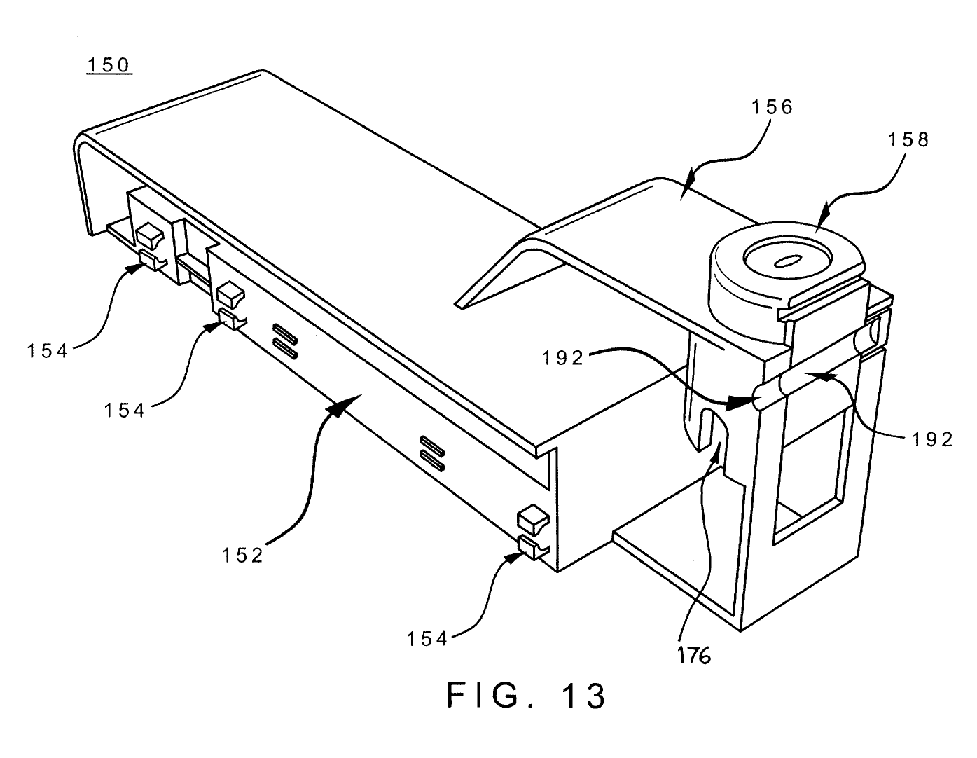

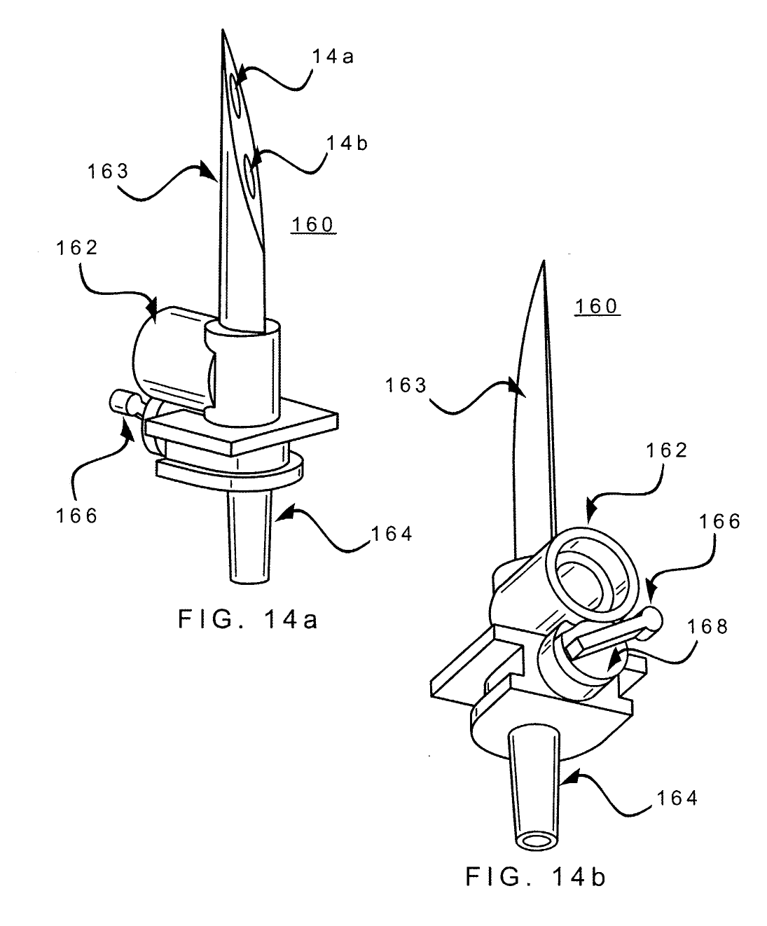

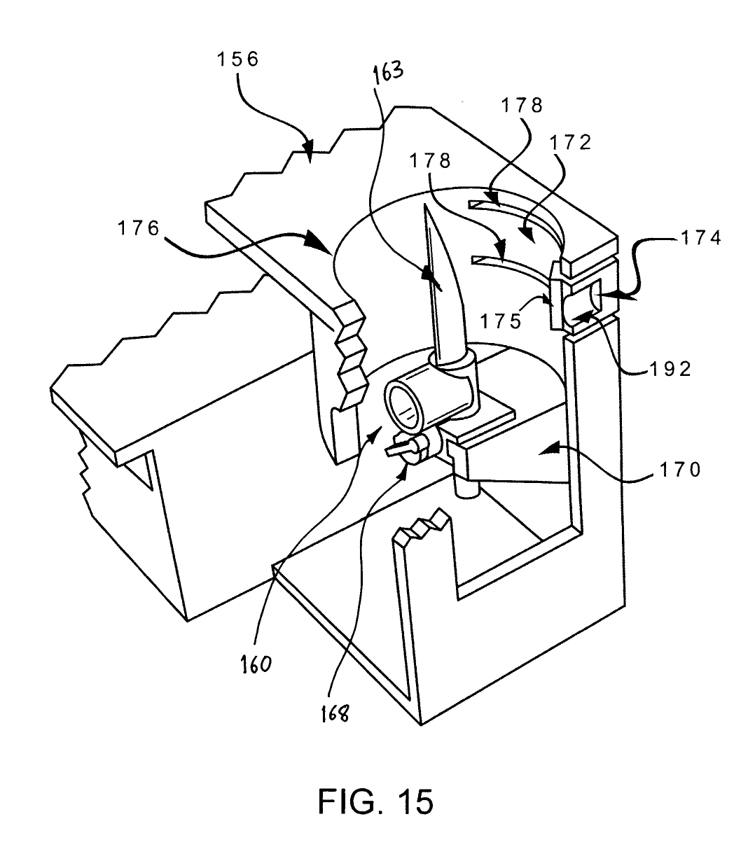

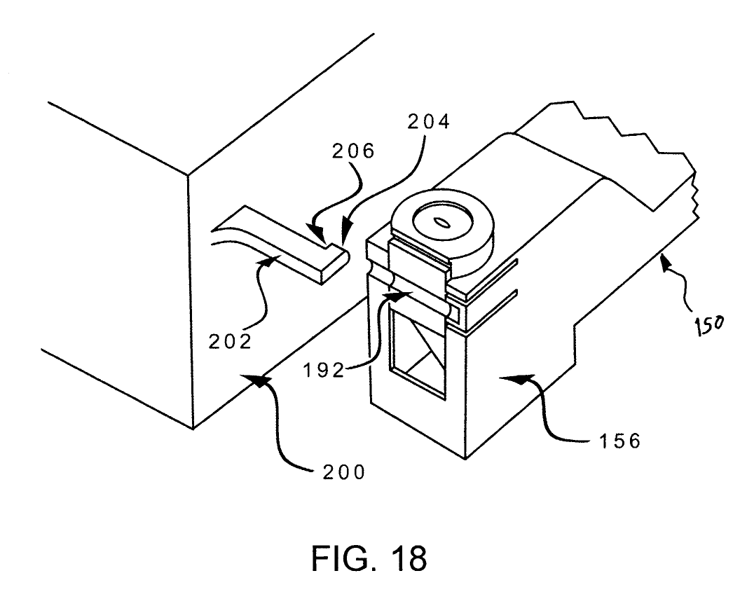

FIG. 13 shows a perspective view of a particular embodiment of a cassette 150 according to the present invention having a pressure plate 152. Pressure plate 152 may include molded snap retainers 154 or other such means of holding delivery conduit 27 or peristaltic tubing (not shown for clarity) in place against the plate. A peristaltic pumping mechanism 56, such as that shown in FIG. 3, may be provided that contacts the tubing and abuts up against the pressure plate 152. Cassette body 156 may contain a cavity 176 (shown in detail in FIG. 15) that receives a slidably mounted spike sheath 158 that is shown in a deployed position over a spike in FIG. 13. Cassette body 156 is constructed so as to allow spike sheath 158 to slide down and expose a spike 163 (shown in detail below with reference to FIGS. 14a and 14b) if cassette 150 is fully engaged with mating surface 200 of the system 36 (as shown in FIG. 18) and is also constructed so as to not allow sheath 158 to slide down if cassette 150 is not fully engaged with surface 200. In particular embodiments of this invention, then, when a new or used cassette 150 is not mounted to mating surface 200, spike sheath 158 will always be deployed to sheath spike 163 and prevent accidental sharps injury. The cassette 150 may then be disposed in a contaminated wastebasket after use with minimized concern about a potential for accidental sharps injury by an exposed spike. A groove 192 may be included on both spike sheath 158 and cassette body 156 to provide clearance for peg 202 of surface 200 (shown in FIG. 18) that fits into groove 192. A breakable fin may be provided on cassette 150 to act as an indicia of use status of cassette 150. The cassette 150 may also be constructed with contoured ridges that provide a better grip for handling the cassette.

FIG. 14a is a perspective view of an embodiment of a spike assembly 160 which may be fitted to cassette 150 and to peristaltic tubing at connector 164. Spike assembly 160 includes spike 163 and may include any or all of air filter housing 162, tapered outlet connector 164 for connection to peristaltic tubing (or other infusion conduit) and lever arm 166 or other like means for actuating a stopcock 168 (FIG. 14b). Spike 163 may include lumens 14a (air venting lumen) and 14b (infusate flow lumen). Air flows via lumen 14a into an infusate container when placed over spike assembly 160 and spiked. This air flow may prevent vacuum buildup inside an infusate container when the container contents are emptied during infusion. Air filter housing 162 may house a filter element (not shown) that filters out airborne disease organisms from the ambient air that flows into the infusate container via lumen 14a. Air filter housing 162 may be designed so as to eliminate the use of an air filter media holder that is traditionally used to contain the air filter media, further reducing parts count and cost of manufacture for the apparatus of the present invention. When lever arm 166 is in the up position as is shown in FIGS. 14a and 14b, stopcock 168 is rotated such that infusate lumen 14b is closed. A closed infusate lumen 14b prevents free flow of residual infusate left in peristaltic and intravenous set tubing and prevents potential entrainment of air emboli into the patient's bloodstream in situations where a used cassette 150 is removed from mating surface 200 while the intravenous set tubing is still connected to a patient.

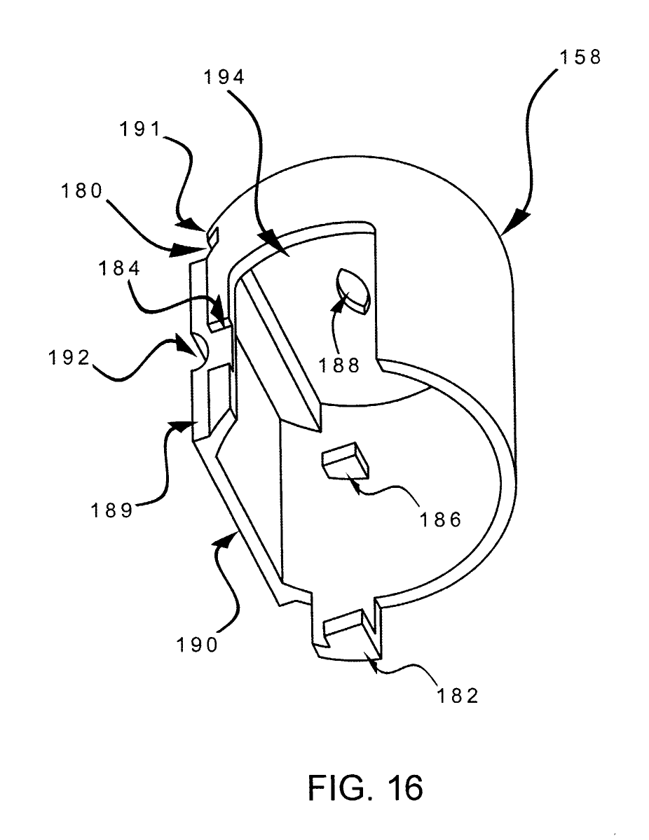

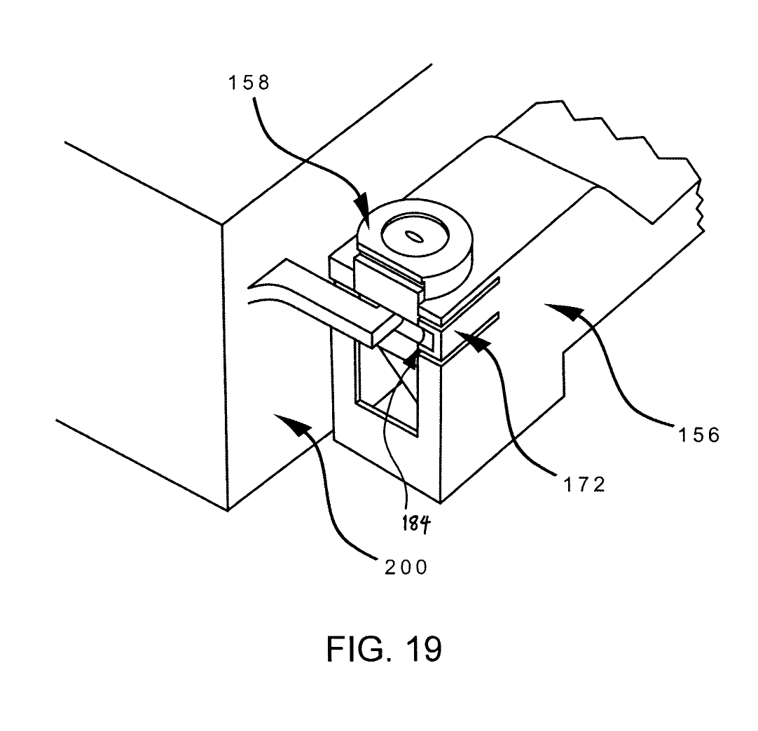

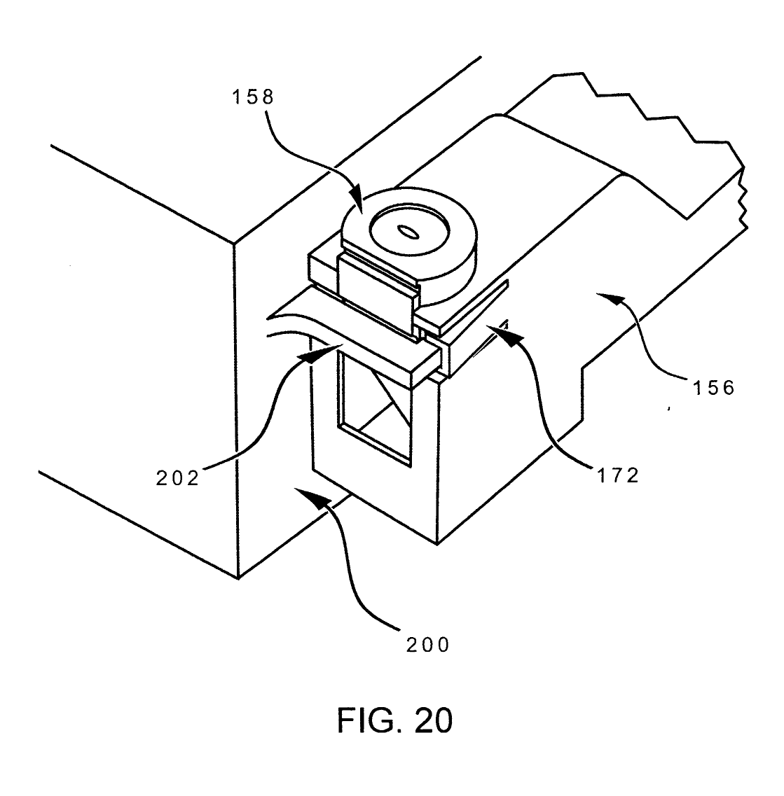

FIG. 15 depicts a cut-out perspective view of cassette body 156 with spike sheath 158 removed. A cavity 176 in cassette body 156 is designed to accept spike sheath 158. Spike assembly 160 is attached to a mounting flange 170 which is incorporated in or itself attached to cassette body 156. Mounting flange 170 holds spike assembly 160 stationary relative to cassette body 156, especially along a vertical axis such that an infusate container may be pushed onto spike assembly 160. A movable member 172 forms part of the wall of cavity 176 and may be made movable by slits 178 cut below and above member 172. Member 172 may have a groove 192 having end 174. Peg 175 on movable member 172 engages with a notch 184 (FIG. 16) or other surface of spike sheath 158. Movable member 172, when in a normal resting, or retracted, position, engages notch 184 (FIGS. 18 and 19) in spike sheath 158 with peg 175 thereby preventing vertical movement of spike sheath 158. When movable member 172 is in a deployed position, peg 175 no longer engages notch 184 (FIG. 20), thereby allowing vertical displacement of spike sheath 158. Movable member 172 is deployed when cassette 150 is substantially engaged with mating surface 200. A peg 202 may be mounted on mating surface 200 in a position so as to deploy movable member 172 by pushing on end 174 of groove 192, when cassette 150 is placed against mating surface 200.

Still referring to FIG. 15, vertical displacement of spike sheath 158 (FIG. 13) allows for each or both of the sheathing and unsheathing of spike 163 and the activation or deactivation of an anti-free flow device. For example, when sheath 158 is in an up position, spike 163 is sheathed by spike sheath 158 and a stopcock 168 is closed thereby preventing free flow of infusion liquid through spike assembly 160. When sheath 158 is in a down position, spike 163 is unsheathed and stopcock 168 is open thereby allowing the flow of infusion liquid through the spike assembly 160.

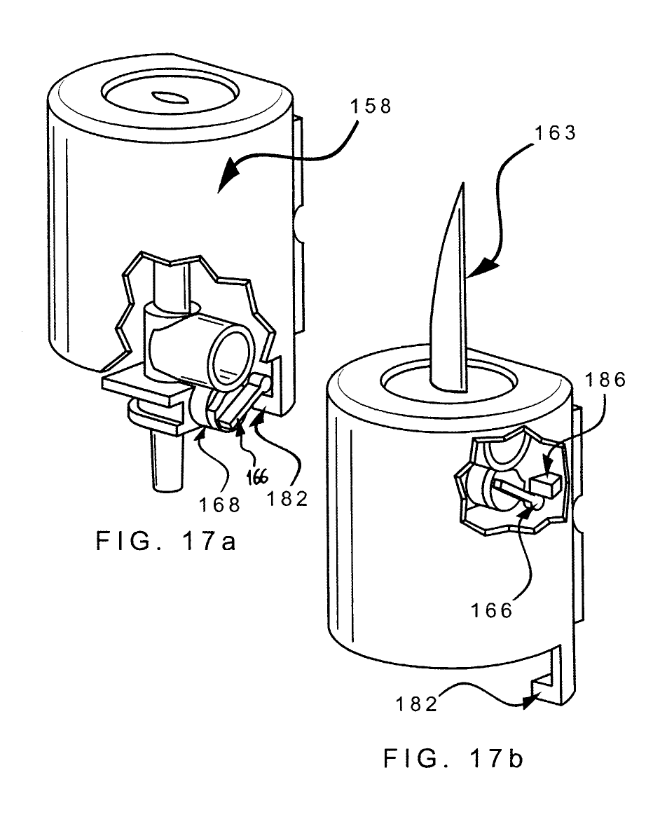

FIG. 16 shows a perspective view of spike sheath 158 which may include portion 190, opening 188 to let spike 163 go through spike sheath 158 and protuberances 182 and 186 that engage with lever arm 166 (FIG. 14b) to close and open stopcock 168 respectively as spike sheath 158 travels up and down (FIGS. 17a and 17b). At the top of portion 190, a step 180 may be provided with a lip 191 which engages with an infusate container holder (not shown).

In a particular embodiment, the infusate container holder engages with step 180 and lip 191 of spike sheath 158 as cassette 150 is engaged to mating surface 200 (FIGS. 18-20). As movable member 172 (FIG. 15) is deployed to allow downwards travel of spike sheath 158, the infusate container holder engages with spike sheath 158 to prevent unplanned downwards travel of the sheath. When the infusate container holder and spike sheath are interlocked, the spike sheath cannot travel down if the infusate container holder is not traveling down. Therefore, in such an embodiment, it is not possible to manually depress the spike sheath and expose the spike, when the cassette is fully engaged to its mating surface.

The infusate container holder is presented to the spike assembly 160 with an inverted infusate container to be spiked when the infusate container holder is moved down against the spike sheath 158. If there is no infusate container in the infusate container holder, downwards travel of the infusate container holder may then expose the spike, posing a risk of a sharps injury. Particular embodiments of the invention check for the presence of an infusate container before allowing downwards travel of the infusate container holder. Checking for the presence of an infusate container may be implemented with sensors, including QAMs 35 (described above with reference to FIG. 11) and/or software or via mechanical means. The invention may also check if the infusate container is valid, e.g., of known origin and quality control and not past its expiration date.

Still referring to FIG. 16, when the infusate container holder is moved down, spike 163 is unsheathed through opening 188 and pierces the infusate container stopper thereby placing lumens 14a and 14b inside the inverted infusate container. The infusate container holder may engage the lip 191 and step 180 of spike sheath 158 such that when the infusate container holder is moved up to unspike an infusate container, the infusate container holder drags spike sheath 158 upwards and re-sheaths spike 163. A cut-out 194 in spike sheath 158 may be included to provide clearance for mounting flange 170 (FIG. 15) when spike sheath 158 travels downwards. A groove 192 on portion 190 (FIG. 16) may be provided with groove 192 of movable member 172 so as to accept edge 204 of peg 202 that is provided with mating surface 200 (FIG. 18). Edges 189 on both sides of portion 190 (FIG. 16) prevent spike sheath 158 from rotating within cavity 176 such that spike sheath 158 is only free to move in a vertical axis. Edges 189 also act as guides for vertical travel of spike sheath 158.

FIG. 17a shows how spike sheath 158 deploys upwards to sheath spike 163 while protuberance 182 engages with lever arm 166 to close stopcock 168 thus preventing flow in infusate lumen 14b of spike 163. FIG. 17b shows how spike sheath 158 retracts downwards to expose spike 163 while protuberance 186 engages with lever arm 166 to open stopcock 168 thus allowing flow in infusate lumen 14b of spike 163.

FIG. 18 shows part of cassette body 156 oriented for engagement with mating surface 200 but not yet contacting the surface. Peg 202 includes edge 204 that slides along groove 192 (FIG. 16) on cassette body 156 and on portion 190 (FIG. 16) of spike sheath 158. Peg 202 may also include a protuberance 206 that abuts against end 174 (FIG. 15) to deploy movable member 172 when cassette 150 is fully engaged with mating surface 200. Protuberance 206 travels along groove 192. A cutout behind protuberance 206 on peg 202 may be included to allow spike sheath 158 to travel downwards without catching on peg 202.

FIG. 19 shows part of cassette body 156 partially engaged with mating surface 200. Edge 204 of protuberance 206 (FIG. 18) of peg 202 is shown engaged in groove 192 on portion 190 (FIG. 16). Spike sheath 158 is still prevented by movable member 172 (FIG. 15) from moving downwards and exposing spike 163. The infusate container holder (not shown) is engaging step 180 and lip 191 of the spike sheath (FIG. 16).

FIG. 20 shows cassette body 156 substantially engaged with mating surface 200 so as to deploy movable member 172 (FIG. 15). Protuberance 206 of peg 202 (FIG. 18) is shown engaged in end 174 of groove 192 (FIG. 15) on cassette body 156. Movable member 172 is deployed allowing spike sheath 158 to be moved downwards and expose spike 163.

It is contemplated that a cassette 150 may be provided as part of a kit of disposable elements for use with an infusate container infusion system such as that described in U.S. patent application Ser. No. 09/324,759, filed Jun. 3, 1999. The cassette may also be provided alone as a disposable or reusable component of an infusate container infusion system. To enhance safety and to prevent accidental injury from spike 163, it is contemplated that the cassette 150 of the present invention may be unpacked from a kit or other packaging or storing material with spike sheath 158 in an up or deployed position so that spike 163 is not exposed. Cassette 150 may be secured to mating surface 200 by an automated mechanism (not shown) or manually. An infusate container (not shown) that is loaded upside down onto an infusate container holder (not shown) may then be positioned in place over the spike assembly 160 and against the spike sheath 158. The infusate container holder is constructed so as to position the infusate container so that the infusate container stopper is aligned and centered with spike sheath 158. The infusate container holder may also engage with lip 191 (FIG. 16) of spike sheath 158 and, when pushed down, drives the infusate container and spike sheath downwards exposing spike 163 and piercing the infusate container stopper. The infusate container holder may be positioned above spike sheath 158 and be manually moved down. As spike sheath 158 travels downwards, lever arm 166 is actuated such that stopcock 168 or other anti-free flow device allows flow of the liquid in the infusate container through infusate lumen 14b. Cassette 158 and the peristaltic and intravenous tubing (IV) may then be purged, the IV tubing connected to an IV catheter, and an infusion process to a patient begun.

At the end of an infusion case, infusate infusion is stopped. The infusate container holder is pulled up and as it moves up it pulls the infusate container up and drags spike sheath 158 along with its lip 191. The upwards travel of spike sheath 158 triggers lever arm 168 closing off infusate lumen 14b. As the infusate container is unspiked, then, spike 163 is resheathed. Once the infusate container is removed, cassette 158 can be disengaged from mating surface 200. Because infusate lumen 14b is closed, none of the residual infusate left in cassette 158 and IV tubing can free flow to a patient still connected to the IV tubing. The IV tubing may then be disconnected from the IV catheter. The intravenous tubing and cassette 150 with the spike assembly 160 may then be discarded in a contaminated wastebasket.

If more than one infusate container is required for a given case, a first infusate container may be unspiked as described above while leaving cassette 150 secured to mating surface 200. Closed infusate lumen 14b prevents aspiration of air into the peristaltic and IV tubing such that there is no need to purge or prime the IV and/or peristaltic tubing again after changing infusate containers. A new infusate container may then be loaded in the infusate container holder and spiked as described above.

I claim:

1. A care system for alleviating patient pain, anxiety and discomfort associated with medical or surgical procedures said system comprising:
a patient health monitor device adapted so as to be coupled to a patient and generate a signal reflecting at least one physiological condition of the patient;
a drug conduit which carries a flow of the drugs from a drug container to the patient;
an infusion pump which effects the flow of drugs through the drug conduit;
a cassette removably interconnected to the infusion pump, wherein a portion of the drug conduit is placed adjacent to the cassette, such that the infusion pump operates in cooperation with the drug conduit to effect the flow of the drugs, and wherein the cassette is adapted to carry a drug container;
a drug delivery controller interconnected with the infusion pump that manages the delivery of one or more drugs from the drug container to the patient via the drug conduit;
a memory device storing a safety data set reflecting safe and undesirable parameters of at least one monitored patient physiological condition; and
an electronic controller interconnected between the patient health monitor, the drug delivery controller, and the memory device storing the safety data set; wherein said electronic controller receives said signal and in response manages the application of the drugs in accord with the safety data set.

2. A cassette for use with an intravenous infusion apparatus including a pumping mechanism which administers an infusate to a patient, wherein said cassette is removably attachable to the intravenous infusion apparatus, and wherein said cassette comprises:
a drug container entry mechanism having an infusate flow lumen, wherein infusate may flow from the container and into the flow lumen;
a pressure plate against which the pumping mechanism may act;
a means for attaching an infusate delivery conduit to the infusate flow lumen such that infusate can flow from the flow lumen into the delivery conduit;
a means for holding the infusate delivery conduit against the pressure plate such that the conduit is acted upon by the pumping mechanism in order to generate a flow of infusate from the container, through the flow lumen, into the delivery conduit, and to the patient; and
an indexing means for aligning the cassette when the cassette is attached to the intravenous infusion apparatus in a manner that allows proper administration of infusate to the patient.

3. A method for administering infusate to a patient, said method comprising the steps of:
attaching an infusate delivery conduit to an infusate flow lumen of a cassette, and aligning the conduit against a rigid pressure plate of the cassette;
attaching the cassette to an intravenous infusion apparatus such that the pressure plate aligns with a pumping mechanism provided with the infusion apparatus;
attaching an infusate container to an infusate container entry mechanism provided with the cassette;
attaching the infusate delivery conduit to a delivery means that is attached to the patient;
purging the infusion flow lumen and the infusion delivery conduit;
initiating the pumping mechanism in order to generate a flow of infusate from the container, through the flow lumen, into the delivery conduit, and to the delivery means attached to the patient; and
modulating the operation of the pumping mechanism based on measured or inferred effects of the infusate on the patient.
